(19) United States
(12) Patent Application Publication
(10) Pub. No.: US 2025/0302780 A1
(43) Pub. Date: Oct. 2, 2025

(54) METHOD OF TREATING PREMATURE EJACULATION IN HUMAN MALES WITHOUT SYMPTOMS OF BENIGN PROSTATIC HYPERTROPHY (BPH) USING TAMSULOSIN 0.2MG QOD WHICH DELAYS EJACULATION BY REDUCING THE RATE OF SECRETION AND TRANSPORT BY SEMINAL VESICLES AND PROSTATE IN THE GENITOURINARY TRACT

(71) Applicant: Solomon Chukwuemeka Awusah, Taylor, MI (US)

(72) Inventor: Solomon Chukwuemeka Awusah, Taylor, MI (US)

(73) Assignee: Wustashire, Taylor, MI (US)

(21) Appl. No.: 18/621,112

(22) Filed: Mar. 29, 2024

(51) Int. Cl. A61K 31/18

(52) U.S. Cl. CPC A61K 31/18

(57) ABSTRACT

In some aspects thereof, the present invention discloses compositions and methods for treating premature ejaculation utilizing tamsulosin at a 0.2 milligram dose administered orally or transdermally every 48 hours. Tamsulosin functions as a selective antagonist of alpha-1a and 1b adrenergic as well as 5HT1A serotonergic receptors implicated in seminal emission pathways. The quantity and frequency of dosing provides an optimal degree of reversible receptor blockade to mildly inhibit sympathetic and serotonergic mediated smooth muscle contraction kinetics governing seminal fluid secretion and transport. This marginally suppresses rate of emission to prolong intercourse without profoundly arresting physiological processes underlying ejaculation.

### FIELD OF THE DISCLOSURE

The present invention relates to a pharmacological composition for the treatment of premature ejaculation in human males. Specifically, the composition targets regulatory mechanisms underlying emission and expulsion phases to delay ejaculation onset and prolong intercourse. The composition aims to selectively modulate certain adrenergic and serotonergic receptor subtypes and associated pathways involved in seminal fluid transport and release.

### BACKGROUND OF THE INVENTION

Premature ejaculation (PE) is characterized by a quick, unintended semen expulsion with minimal sexual stimulation. Ejaculation involves a complex physiological process consisting of emission and expulsion phases that lead to semen release. Research suggests the existence of a spinal ejaculation generator (SEG) that integrates signals to regulate ejaculation.

In the emission phase of ejaculation, sympathetic nervous system as well as serotonergic stimulation trigger smooth muscle contraction, causing sperm and seminal fluid to empty into the prostatic urethra. Physical or sensory stimuli activate this pathway. During the emission phase, the ampullae of the vas deferens and seminal vesicles eject sperm and seminal fluid into the ejaculatory ducts, which then drain into the prostatic urethra where prostatic secretions also add to the descending seminal fluid. The accumulation of seminal fluid in the prostatic urethra then triggers the activation of the SEG, initiating the expulsion phase.

Chemosensory receptors in the prostatic urethra transmit signals to determine whether to proceed along the ejaculatory or micturition pathways. The SEG integrates these inputs and relays signals to brainstem areas that regulate ejaculation, which are on the left dorsolateral pontine tegmentum and right ventrolateral pontine tegmentum. The ejaculation control centers cause the relaxation of the external urethral sphincter as well as the contraction of the pelvic floor muscles during ejaculation through direct projections to pelvic floor motor neurons. These rhythmic muscle contractions facilitate outward seminal fluid transport.

In the emission phase, smooth muscle contractions can occur through sympathetic nervous system activation of alpha-1a and 1b adrenoreceptors as well as serotonergic activation of 5HT1A receptors in the genitourinary tract. As more receptors are activated, the secretory response intensifies. While this emission alone may not account for the full volume of the final ejaculate, it provides adequate stimulation to trigger the expulsion reflex. Subsequent skeletal muscle contractions of the pelvic muscles aid in transporting this seminal fluid; thereby determining the final ejaculate volume. Blocking alpha-1a and 1b adrenoreceptors as well as 5HT1A serotonergic receptors in the genitourinary tract can inhibit or weaken emission contractions (Quaresma et al, 2019). Extensive blockade may prevent emission almost entirely, while partial blockade slows emission and transport; thereby, delaying ejaculation.

Smooth muscle contraction relies on calcium signaling pathways triggered by neurotransmitters or membrane depolarization. Neurotransmission requires norepinephrine release or the presence of a viable agonist to activate receptors coupled to downstream signaling cascades, inducing intracellular calcium release and muscle contraction. The more receptors that are activated, the stronger the response. Membrane depolarization enables myogenic contraction in response to mechanical factors like pressure or stretching. By opening calcium channels, depolarization elevates intracellular calcium levels to activate contraction. The robust skeletal muscle contractions during expulsion likely stretch adjacent smooth muscles, evoking this depolarization-mediated mechanism.

Research shows that alpha-1a and 1b adrenergic as well as 5HT1A serotonergic activities in the genitourinary tract facilitate ejaculation. Prolonged agonist exposure can precipitate premature ejaculation, while antagonist studies reveal delayed or absent ejaculation depending on blockade extent. As emission depends on receptor activation, blockade inhibits this process. Extensive blockade may prevent emission and cause anejaculation, while partial blockade merely slows emission and transport which delay ejaculation without significantly affecting the overall ejaculate volume.

Unfortunately, treatment options specifically approved by the FDA for premature ejaculation remain limited in the United States. As of now, the only FDA-approved prescription medication for premature ejaculation is a topical anesthetic called Promescent. However, several other pharmacologic therapies are commonly prescribed off-label to help delay ejaculation, including antidepressants like selective serotonin reuptake inhibitors (SSRIs), topical anesthetics like lidocaine, and phosphodiesterase type 5 inhibitors (PDE5Is) like sildenafil. Using medications off-label means they have not undergone clinical trials and formal FDA approval processes to demonstrate safety and efficacy specifically for treating premature ejaculation. This off-label status illustrates that premature ejaculation remains an underserved condition lacking dedicated treatment development.

It is therefore an object of the current invention to develop an effective pharmacological treatment for premature ejaculation that does not significantly impact ejaculate volume or cause other ejaculatory side effects.

### SUMMARY

One aspect of the present disclosure relates to a pharmacological composition containing 0.2 milligrams of tamsulosin which is administered orally every 48 hours (every other day) for premature ejaculation treatment in non-BPH male patients. Tamsulosin is the active compound functioning as an antagonist of specific adrenergic and serotonergic receptors involved in seminal emission. This selective receptor antagonist activity elicits relaxation of smooth muscles regulating ejaculatory physiology, attenuating sympathetic and serotonergic stimulation of seminal fluid secretion and transport. The quantity and dosing frequency of tamsulosin were precisely calculated to achieve partial blockade sufficient for prolonging intercourse by modestly reducing the rate of seminal vesicle and prostate secretions. However, the degree of antagonism remains below levels that could promote adverse sexual side effects like ejaculation failure. Thus, the unique dosage form enables effective treatment of premature ejaculation while preserving normal ejaculate volumes.

Another aspect of the disclosure relates to a therapeutic technique for premature ejaculation utilizing the 0.2 milligram tamsulosin composition every other day. This method involves oral self-administration of the tailored tamsulosin formulation to selectively impede activation of genitourinary smooth muscle contractions contributing to rapid emission in non-BPH male patients. Outcomes demonstrate that this dosage regimen significantly extends latency and intercourse duration by mildly inhibiting sympathetic and serotonergic stimulation of ampullary, seminal vesicular, and prostatic secretion as well as damping external urethral sphincter reflexes. However, the calculated quantity and frequency prevent complete blockade of the physiologic ejaculatory reflex. Consequently, the method enables treatment of premature ejaculation while maintaining largely preserved semen parameters and minimal risks of adverse ejaculatory side effects that could undermine therapeutic goals.

### BRIEF DESCRIPTION OF THE DRAWINGS

FIG. 1 illustrates how tamsulosin binds to active sites.

FIG. 2 illustrates the addition of a reversible competitive antagonist to a non-vascular smooth muscle neuromuscular junction.

FIG. 3 is a dose-response curve quantifying the relationship between assigned tamsulosin dosage and projected probability of undesirable ejaculatory side effects.

FIG. 4 illustrates test data on calculated risk of adverse ejaculatory side effects for different dosages.

### DETAILED DESCRIPTION

To objectively determine an effective tamsulosin dose for treating premature ejaculation in patients without benign prostatic hypertrophy (BPH), a focused analysis of relevant well designed research studies that examined the abnormal ejaculation side effects of tamsulosin in BPH treatment based on combined data derived from multiple sources was conducted (www.accessdata.fda.gov, n.d.; Soliman et al, 2023; Shim et al., 2016). Abnormal ejaculation is defined as anejaculation or significantly reduced ejaculate. These studies focused on evaluating tamsulosin treatment outcomes in patients with BPH and lower urinary tract symptoms (LUTS). The analysis leveraged the identical mechanism of action of tamsulosin in both BPH/LUTS and premature ejaculation with respect to non-vascular smooth muscle relaxation in the genitourinary tract.

As an avid alpha-1a and 1b adrenoreceptor and 5HT1A serotonergic receptor antagonist, tamsulosin exhibits a shared mechanism of action in treating both benign prostatic hypertrophy (BPH) and premature ejaculation. In both conditions, tamsulosin targets the non-vascular smooth muscles of the genitourinary tract, specifically those in the seminal vesicles and prostate.

In BPH, the enlargement of the prostate gland can lead to compression of the urethra, causing lower urinary tract symptoms (LUTS) such as weak urine stream, incomplete emptying, and frequent urination. Tamsulosin acts by relaxing the smooth muscles in the prostate and bladder neck, thereby reducing the constriction of the urethra and alleviating LUTS.

Similarly, in premature ejaculation, the contraction of smooth muscles in the seminal vesicles and prostate plays a crucial role in the emission and expulsion of semen. Tamsulosin, by binding to the alpha-1a and 1b adrenergic as well as 5HT1A serotonergic receptors in these tissues, promotes smooth muscle relaxation, which in turn slows down the ejaculatory process and increases the time to reach climax.

Given the identical mechanism of action in both conditions, it is reasonable to expect that a tamsulosin dose proven effective in treating BPH symptoms would also demonstrate efficacy in managing premature ejaculation. The level of smooth muscle relaxation required to relieve most urethral obstruction in BPH is by deduction considered to be higher than that needed to delay ejaculation in premature ejaculation based on observed increased ejaculatory dysfunction risk associated with some effective BPH treatment occurring at high doses.

As such, if a specific lower dose of tamsulosin with minimal adverse ejaculatory risk can effectively induce sufficient smooth muscle relaxation to improve LUTS in BPH, it should also be capable of providing sufficient smooth muscle relaxation required to prolong the time to ejaculation in premature ejaculation. This concept supports the extrapolation of dose efficacy data from BPH studies to inform the selection of an optimal tamsulosin dose for the treatment of premature ejaculation.

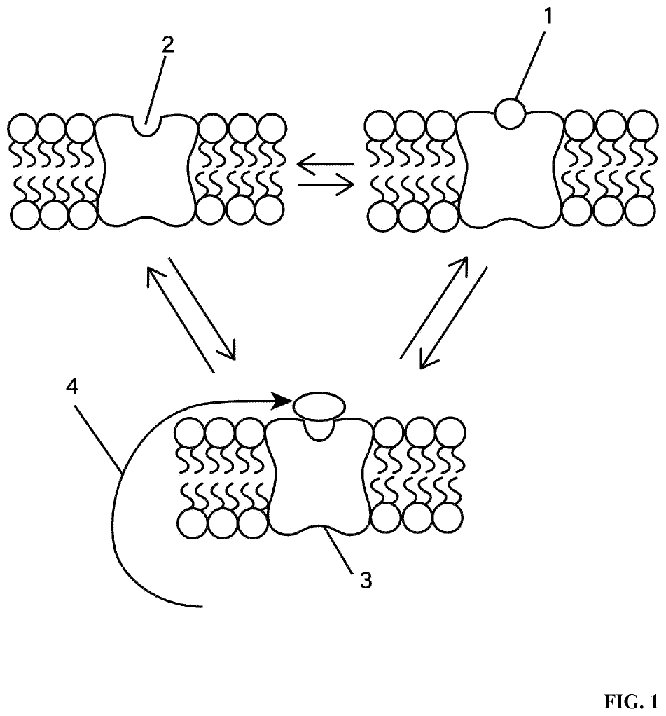

The exemplary embodiment according to FIG. 1 provides a schematic demonstration of the mechanism underlying tamsulosin's therapeutic effects in relaxing genitourinary smooth muscle tone. The illustration depicts alpha-1a and 1b adrenergic or 5HT1A serotonergic receptors 3 embedded within seminal vesicles, prostate gland or prostatic urethral smooth muscle cell membranes. These receptors possess active sites 2 that bind the endogenous catecholamine norepinephrine or a viable agonist like serotonin. Norepinephrine or serotonin binding triggers intracellular signaling cascades culminating in smooth muscle contraction. However, as shown in the figure, tamsulosin 1 can also bind to these same alpha-1a and 1b adrenergic as well as 5HT1A serotonergic active sites, occupying the niche and preventing norepinephrine or serotonin binding. This competitive antagonism blocks activation of receptors and downstream contraction.

Noteworthy, Tamsulosin exhibits high affinity for the alpha-1a and alpha-1b adrenoreceptor subtypes as well as 5HT1A serotonergic receptor subtype located in the genitourinary tract. This subtype selectivity allows tamsulosin to effectively relax the non-vascular smooth muscles of the seminal vesicles and prostate. Additionally, the binding interaction between tamsulosin and its receptors is reversible rather than permanent 4. This enables the medication to dissociate from the receptors after a period, restoring their availability for natural neurotransmitter signaling. Reversibility is essential from a safety perspective and prevents permanent disruption of normal physiologic control pathways regulating smooth muscle contractility.

This illustrated mechanism of FIG. 1 underlies how delivering Tamsulosin proves efficacious in managing Premature Ejaculation. 5HT1A along with alpha-1a and 1b receptor activities facilitate contraction of seminal vesicles, prostate, urethral bulb and ejaculatory smooth muscles essential for emission and expulsion of seminal fluid. By binding and blocking activation of these receptors in the male reproductive tract 3, tamsulosin impedes sympathetic and serotonergic stimulation of the ejaculatory reflex. This hampers rapid seminal fluid secretion and transport while also dampening expulsion phase external urethral sphincter responses. Consequently, tamsulosin extends the time until climax, preventing the minimal genital stimulation triggering premature ejaculation.

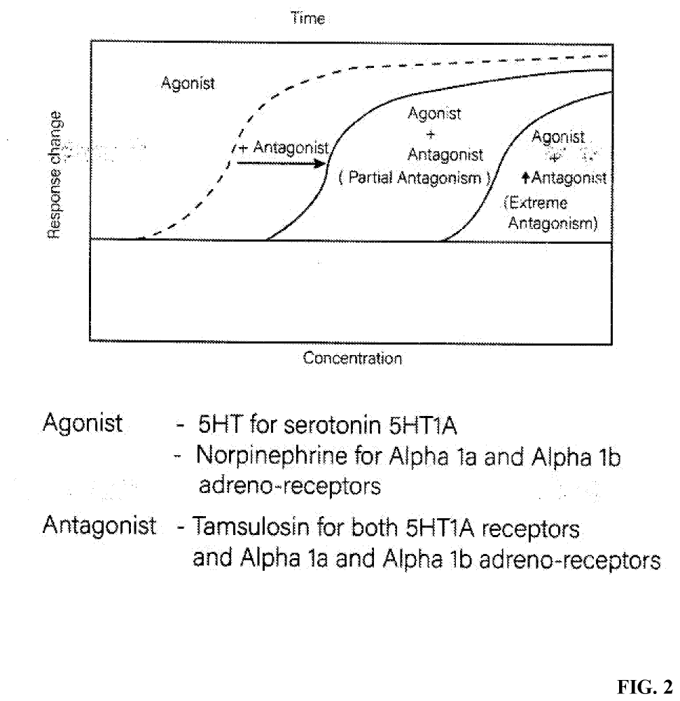

On the other hand, the FIG. 2 illustrates the addition of a reversible competitive antagonist to a non-vascular smooth muscle neuromuscular junction. It is a graphical representation of the impact of escalating concentrations of the pharmacological agent tamsulosin, functioning as a reversible competitive antagonist at the non-vascular smooth muscle neuromuscular junction. In this example, the neuromuscular junction would normally transmit signals from endogenous agonists such as norepinephrine or serotonin (5-HT)—which can also activate alpha-1a and 1b adrenergic or 5-HT1A serotonergic receptors to elicit non vascular smooth muscle contraction. As referenced earlier in the detailed description, tamsulosin exhibits a high affinity for these receptor subtypes as an antagonist.

The graph illustrates extensive antagonism that occurs with substantially elevated tamsulosin levels, which is sufficient to almost completely block agonist-induced activation of post-synaptic receptors. This extensive inhibition of alpha-1a and 1b as well as 5-HT1A receptor stimulation prevents non-vascular seminal vesicle and prostate smooth muscle contraction required for seminal fluid secretion. Consequently, higher concentrations of tamsulosin can profoundly arrest emission, resulting in anejaculation and ejaculation failure—unacceptable side effects compromising therapeutic goals for premature ejaculation.

Conversely, the graph also demonstrates partial antagonism with moderately increased, but not extensive tamsulosin concentrations. This measured degree of blockade only dampens, but does not profoundly stop, non-vascular smooth muscle contraction regulating seminal fluid release. Therefore, as highlighted in the discussion, calculated dosing that produces partial antagonism can appropriately slow emission to delay climax without completely halting secretion or preventing ejaculation. This mechanism enables treating premature ejaculation while avoiding ejaculatory dysfunction. Moreover, higher tamsulosin doses that inherently induce extensive receptor blockade may sometimes be desirable for maximizing smooth muscle relaxation in benign prostatic hypertrophy therapy.

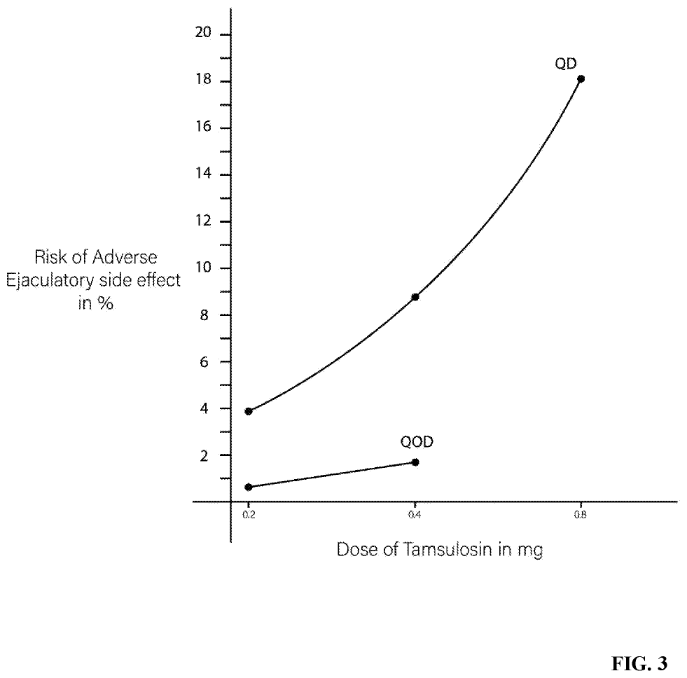

The illustrative embodiment according to FIG. 3 provides a visualized dose-response curve quantifying the relationship between assigned tamsulosin dosage and projected probability of undesirable ejaculatory side effects including anejaculation and markedly reduced seminal emission. The graph plots administered tamsulosin quantity in mg on the x-axis against the calculated percentage risks of these dysfunctional sequelae on the y-axis.

Two distinct dose-risk curves are shown corresponding to conventional once daily administration (QD) or alternate day timetable (QOD). At equivalent dosages, the QOD regimen demonstrates a consistently lower risk profile compared to QD which affirms Tamsulosin's capacity for schedule manipulation to enhance tolerability independent of dosage strength. For both plots, the quantitative mapping substantiates that higher doses predictably drive higher event risks reflecting heightened receptor blockade. However, diminishing dose illustrates almost linear risk decreases suggesting potential opportunity to favorably constrain side effect likelihood through tailored titration.

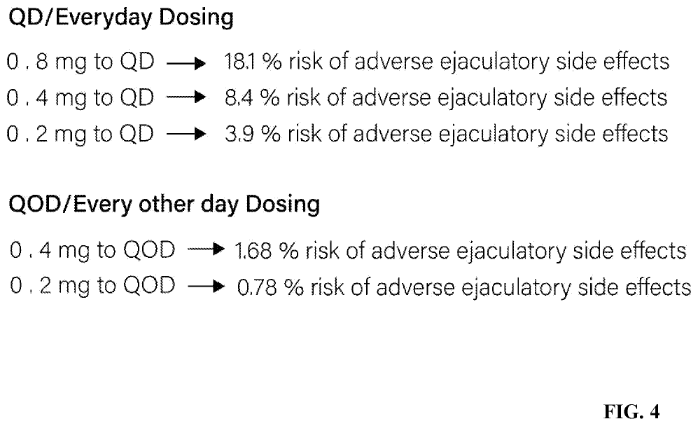

Specifically, halving tamsulosin 0.8 mg QD dose to 0.4 mg QD reduces projected abnormal or adverse ejaculatory risk by 53.6% from 18.1% to 8.4%. A further 50% dose reduction to 0.2 mg QD expects an approximate 2.15-fold drop in abnormal ejaculatory risk likelihood (www.accessdata.fda.gov, n.d.; Shim et al., 2016). This almost linear risk gradient supports dose modulation as a means to safely minimize therapy interference with physiological ejaculatory reflexes. Converting the dosing frequency for 0.4 mg from QD to QOD additionally delivers 5-fold abnormal ejaculatory risk reduction despite consistent dose (Soliman et al., 2023). The reversible competitive antagonism mechanism of action of tamsulosin agrees with this observation which reflects decreased ejaculation risk with time using intermittent dosing manipulation. Hence, the visualized quantitative landscape provided by FIG. 4 based on direct proportionality substantiates conclusions that 0.2 mg QOD regimen offers optimal efficacy-safety balance exploiting additive benefits of dose and schedule manipulation to constrain ejaculation dysfunction risks close to or below 1%.

Further, the FIG. 4 illustrates test data on calculated risk of abnormal or adverse ejaculatory side effects at different dosages. The figure shows a summary analysis of mathematical projections estimating dose-dependent risks of undesirable ejaculatory side effects with tansulosin treatment based on the focused analysis of the aforementioned studies (www.accessdata.fda.gov, n.d.; Soliman et al, 2023; Shim et al., 2016). Quantitative data points are shown for tamsulosin at doses of 0.8 mg, 0.4 mg, and 0.2 mg taken either on a daily (QD) basis or every other day (QOD). The percentages signify the predicted probability of adverse events like anejaculation or substantially reduced ejaculatory volume occurring as treatment consequences.

As contextualized in the results and calculation details, higher doses of daily tamsulosin dosing correlate with greater rates of ejaculatory abnormalities given more pronounced antagonism of receptors governing emission. For example, the chart illustrates 0.8 mg daily was associated with 18.1% risk compared to 8.4% for the 0.4 mg daily dose as evidenced by referenced trials. Further halving the dose to 0.2 mg daily projects about 3.9% probability of these dysfunctional side effects (Shim et al., 2016). This dose-response trend reinforces the ability to titrate blockade magnitude, and the associated impact on ejaculatory function, based on quantity.

Additionally, the graphical data demonstrates that modifying schedule rather than just the amount enables further fine-tuning of the ejaculatory effects. Using observations made by Soliman et al. as a guide, converting the 0.4 mg daily dose to 0.4 mg every other day would reduce the risk of ejaculatory sequelae by 5-fold from 8.4% to 1.68% per integrated study findings. Extrapolating this response based on direct proportionality, the chart displays 0.2 mg QOD projection to around a 0.78% risk of ejaculatory abnormalities. According to Soliman et al., the therapeutic efficacy of once-daily (QD) dosing of tamsulosin is clinically similar to the alternating day dosing but with fewer side effects. This indicates that tamsulosin's receptor residency time is longer than its documented plasma half-life of 9 to 13 hours. Tamsulosin 0.2 mg QD is effective for treating mild to moderate BPH symptoms (shim et al., 2016). If this dose can achieve the necessary smooth muscle relaxation for BPH, it should also be sufficient for treating premature ejaculation. Zhang et al. found that tamsulosin 0.2 mg QD increased intravaginal ejaculation latency time (IELT) by 1.14 minutes with a 2.6% risk of adverse events after 8 weeks. Therefore, it is anticipated that alternating day therapy will yield similar effects while reducing the risk of adverse ejaculatory events to around 1% or lower.

The observation reported by Zhang et al. differs from the proposed method of this invention given that with this invention, patients with symptoms of BPH are excluded from the targeted treatment group. This is because some BPH patients with a higher degree of hypertrophy would require a higher dose of tamsulosin for symptomatic relief and therefore, would be expected to still show a low IELT with a lower dose. The dosing and response projections provided in this document show supporting evidence that 0.2 mg QOD regimen strikes an optimal balance between maximizing premature ejaculation treatment efficacy in patients without symptoms of BPH through modest alpha-1a and 1b as well as 5HT1A receptor antagonism to prolong intercourse while mitigating chances of therapy-induced ejaculatory dysfunction. The visualized risk quantification substantiates the conclusions that this tailored tamsulosin regimen and schedule allows clinically meaningful benefits with minimal detriment to normal sexual function.

In certain aspects, the 0.2 mg tamsulosin composition could be integrated into a sustained-release capsule formulation to further extend the effective treatment window after each dose and maintain steady state plasma levels.

In certain aspects, a transdermal patch harboring metered tamsulosin 0.2 mg delivery over a 48-hour timeframe may offer convenient administration and controlled absorption profiles suited for every other day treatment.

In certain aspects, as smoking and antioxidant deficiencies correlate with premature ejaculation incidence, the therapy could be co-packaged with patches, gums, or supplements targeting lifestyle improvement.

In certain aspects, compositions can embed the 0.2 mg dosage into a rapid dissolve sublingual platform for mucosal absorption in order to further hasten PE symptom relief onset during intercourse.

In certain aspects, combining tamsulosin with agents like sildenafil, topical anesthetics, SSRIs or counseling enables tailored multi-modal PE regimens personalized to patient needs.

In certain aspects, the treatment technique allows intermittent use; temporarily discontinuing tamsulosin for desired fertility cycles to preserve uninhibited reproductive capacity.

### INDUSTRIAL APPLICATION

The disclosed invention harboring both a therapeutic composition and technique for managing premature ejaculation bears extensive applications across the medical, pharmaceutical, and healthcare industries. The tailored tamsulosin formulation and pioneering receptor-targeted dosing regimen could provide an approved, science-backed premature ejaculation therapy that can potentially fill a major drug development void. Generating a low-cost oral medication specifically designed to safely delay climax with marginal impact on sexual experience can spur a new self-treatment market segment. Industrial-scale production of the 0.2 mg tamsulosin pill coupled with clinical validation studies supporting proprietary claims may enable lucrative market exclusivity.

### REFERENCES

[1] Alwad, A., Breyer, B & Lue, T. F. (2015). Normal Male Sexual Function: Emphasis on orgasm and Ejaculation. Fertil Steril. 104-(5): 1051-1060.
[2] Revenig, L., Leung, A., & Hsiao, W. (2014). Ejaculatory Physiology and Pathophysiology: Assessment and Treatment in Male Infertility. Transl Androl Urol. 3(1): 41-49.
[3] Carro-Juarez, M., & Rodriguez-Manzo, G. (2008). The Spinal Pattern for Ejaculation. Brain Res Rev. 58(1):106-20.
[4] Chehensse, C., Facchinetti, P., et al. (2017). Human Spinal Ejaculation Generator. Ann Neurol. 81(1):35-45.
[5] Allard, J., Truitt, W., McKenna, K., & Coolen, L. (2005). Spinal Cord Control of Ejaculation. World J Urol. 23(2):119-26.
[6] McKenna, K. E., Chung, S. K., & McVary, K. T. (1991). A Model for the Study of Sexual Function in anesthetized male and female rats. Am J. Physiol. 261(5 Pt 2): R1276-85.
[7] Deckmann, K., & Kummer, W. (2016). Chemosensory epithelial cells in Urethra: sentinels of the urinary tract. Histochem Cell Biol. 146(6): 673-683.
[8] Deckmann, K., Fillipski, K., Krasteva-Christ, G. et al. (2014). Bitter triggers Acetylcholine release from Polymodal Urethral Chemosensory Cells and Bladder Reflexes. Proc Natl Acad Sci USA. 111(22): 8287-92
[9] Kummer, W., & Deckmann, K. (2017). Brush cells, the newly identified gatekeepers of the urinary tract. Curr Opin Urol. 27(2):85-92.
[10] Huynh, H. K., Willemsen, A. T. M., Lovick, T. A., & Holstege, G. (2013). Pontine control of ejaculation and female orgasm. J Sex Med. 10 (12): 3038-48.
[11] Clinton Webb, R. (2003). Smooth Muscle Contraction and Relaxation. American Physiology Society Journal; published online 1 Dec. 2003.
[12] Akin, Y., Gulmez, H., Ates, M., Bozkurt, A., & Nuhoglu, B. (2013). Comparison of Alpha Blockers in Treatment of Premature Ejaculation: A pilot Clinical Trial; Iran Red Crescent Med J. 15(10): e13805
[13] Quaresma, B. M. C. S., Pimenta, A. R., Santos da Silva, A. C., et al. (2019). Revisiting the Pharmacodynamic uroselectivity of a¹ Adrenergic receptor antagonists. J. Pharmacol Exp Ther. 371(1) 106-112.
[14] Hsieh, J., Liu, S., Chang, H., et al. (2011). The Activation of peripheral 5-HT1A receptors can inhibit seminal vesicle contractions: an in vivo animal study. Urology. 78 (2) 376-9.
[15] Mittra, S., Malhorta, S., Naruganahalli, K. S., & Churgh, A. (2007). Role of peripheral 5-HT1A receptors in detrusor over activity associated with partial bladder outlet obstruction in female rats. Eur J Pharmacol. 561(1-3) 189-93.
[16] Narayan, P., & Tunuguntla, H. S. G. R. (2005). Long-term Efficacy and Safety of Tamsulosin for Benign Prostatic Hyperplasia: Rev Urol. 7(supple 4): S42-S48.
[17] Lee, C., & Kuo., H. (2017). Pathophysiology of benign prostate enlargement and lower urinary tract symptoms: Current concepts. Tzu Chi Med J. 29(2):79-83.
[18] Dhingra, N., & Bhagwat, D. (2011) Benign Prostatic hyperplasia: An overview of Existing Treatment. Indian J Pharmacol. 43(1):6-12.
[19] Saleh, R., Majzoub, A., & El-Hamd, M. A. (2021). An update on the treatment of premature ejaculation: A systematic review. Arab J Urol. 19(3):281-302.
[20] https://www.accessdata.fda.gov/drugsatfda_docs/label/2009/020579s026lb1.pdf
[21] Soliman, M. G., Al-Ghadeer, M. R., Al-Shabaan, H. R., Al-Hamrani, A. H., & Al-Ghadeer, H. A. (2023). Evaluation of Intermittent Tamsulosin in treating symptomatic patients with benign prostatic hyperplasia; Urol Ann. 15(1) 43-47.
[22] Shim, S. R., Kim, J. H., Chang, I. H., et al. (2016). Is Tamsulosin 0.2 mg Effective and Safe as a First-Line treatment compared with Other Alpha Blockers? A Meta-Analysis and a Moderator Focused Study; Yonsel Med J. 57(2) 407-418.
[23] Kim, S. W., Lee, W. C., Kim, M. T., et al. (2013). Effects of low dose tamsulosin on sexual function in patients with lower urinary tract symptoms suggestive of benign prostatic hyperplasia. Korean J. Urol. 54: 697-702.
[24] Park, C. H., Chang, H. S., Oh, B. R., et al. (2004). Efficacy of low dose tamsulosin on lower urinary tract symptoms suggestive of benign prostatic hyperplasia: A non-blind multicentre Korean study. Clin Drug Investig. 24: 41-7.
[25] Elsaied, M. A., Mostafa, T., & Abdel-Hamid, I. A. (2016). The Drug Treatment of Delayed Ejaculation. Transl Androl Urol. 5(4): 576-591.
[26] Capogrosso, P., Serino, A., Ventomiglia, E., et al. (2015). Effects of Solidosin on sexual function-realistic picture from the everyday clinical practice. Andrology: 3(6): 1076-81.
[27] Hisasue, S., Furuya, R., Ito, N., et al. (2006). Ejaculatory disorder caused by alpha-1 adrenoreceptor antagonists is not retrograde ejaculation but a loss of seminal emission. Int. J. Urol. 13(10): 1311-6.
[28] Zhang, C., Li, B., Zhang, Y. et al. (2014). Clinical study of lifelong premature ejaculation with paroxetine hydrochloride and tamsulosin. Transl Androl Urol 2014; 3(S1): AB151.doi: 10.3978/j.issn.2223-4683. 2014.s151
[29] Copeland, R. A., Pompliano, D. L., & Meek, T. D. (2006). Drug-target residence time and its implications for lead optimization. Nature Review Drug Discovery: 5(9), 730-9.

What is claimed is:

1. A pharmaceutical composition for treating premature ejaculation comprising: 0.2 milligrams of tamsulosin as the sole active ingredient, formulated into one of an oral or transdermal dosage form.

2. The composition of claim 1, wherein the 0.2 milligrams of tamsulosin functions as a selective antagonist of alpha-1a and 1b adrenergic as well as 5HT1A serotonergic receptors localized to reproductive organs and structures facilitating seminal fluid secretion and transport.

3. The composition of claim 1 formulated as a tablet, capsule, film, or orally disintegrating tablet adapted for oral administration every 48 hours.

4. The composition of claim 1 further comprising standard pharmaceutical excipients including diluents, binders, disintegrants, flavoring, coloring, pH modifiers, or digestive aids.

5. The composition of claim 1, exhibiting pharmacokinetic properties aligned to maintain marginally-inhibitory tamsulosin levels only sufficient to prolong latency without profoundly halting the physiological processes underlying seminal emission.

6. The composition of claim 1, wherein the quantity and potency provide low risk of therapy-associated ejaculation failure or seminally devoid orgasm meeting clinical benchmarks for premature ejaculation treatment safety.

7. The composition of claim 1, adapted for incorporation into a sustained-release platform or transdermal patch continuously releasing 0.2 milligrams total over approximately a 48-hour duration.

8. The composition of claim 1 packaged with validated patient instructions directing oral self-administration every 48 hours for managing premature ejaculation symptoms with minimal adverse ejaculatory side effects in patients without symptoms of Benign Prostatic Hyperplasia (BPH), wherein the exclusion of patients with symptoms of BPH from the target treatment group is based on the observation that some BPH patients with a higher degree of hypertrophy require a higher dose of tamsulosin for symptomatic relief and are expected to still show a low Intravaginal Ejaculatory Latency Time (IELT) with a lower dose of tamsulosin.

9. A method for treating premature ejaculation comprising administering to a subject in need thereof a pharmaceutical composition comprising: 0.2 milligrams of tamsulosin as the sole active agent formulated into an oral or transdermal dosage form.

10. The method of claim 9, wherein the pharmaceutical composition is formulated for oral administration as a tablet, capsule, film, or orally disintegrating tablet adapted to deliver the 0.2 milligrams dosage of tamsulosin every 48 hours.

11. The method of claim 9, wherein the pharmaceutical composition is adapted for continuous transdermal delivery of 0.2 milligrams of tamsulosin over approximately a 48-hour duration.

12. The method of claim 9, wherein premature ejaculation treatment efficacy and safety benchmarks are defined as prolonging intercourse prior to climax while maintaining normal seminal parameters and orgasmic function with low risk of therapy induced ejaculatory dysfunction such as anejaculation or significantly reduced ejaculatory volume.

13. The method of claim 9, wherein the pharmaceutical composition exhibits pharmacokinetic properties calibrated to provide sufficient tamsulosin levels for modest alpha-1a and 1b adrenergic as well as 5HT1A serotonergic receptor inhibition in the male genitourinary system.

14. The method of claim 9, wherein instructions direct patient self-administration of the pharmaceutical composition orally or transdermally every 48 hours to safely manage premature ejaculation symptoms.

15. A method for treating premature ejaculation in human males without symptoms of Benign Prostatic Hyperplasia (BPH), comprising administering to a subject in need thereof a pharmaceutical composition comprising 0.2 milligrams of tamsulosin as the sole active agent, formulated into an oral or transdermal dosage form, wherein the administration is performed every 48 hours.

16. The method of claim 15, wherein the pharmaceutical composition is formulated for oral administration as a tablet, capsule, film, or orally disintegrating tablet.

17. The method of claim 15, wherein the pharmaceutical composition is adapted for continuous transdermal delivery of 0.2 milligrams of tamsulosin over approximately a 48-hour duration.

18. The method of claim 15, wherein the treatment prolongs intercourse prior to climax while maintaining normal seminal parameters and orgasmic function with a low risk of therapy-induced ejaculatory dysfunction such as anejaculation or significantly reduced ejaculatory volume.

19. The method of claim 15, wherein the pharmaceutical composition exhibits pharmacokinetic properties calibrated to provide sufficient tamsulosin levels for modest alpha-1a and 1b adrenergic as well as 5HT1A serotonergic receptor inhibition in the male genitourinary system.

20. The method of claim 15, wherein the exclusion of patients with symptoms of BPH is based on the observation that some BPH patients with a higher degree of hypertrophy require a higher dose of tamsulosin for symptomatic relief and are expected to still show a low Intravaginal Ejaculatory Latency Time (IELT) with a lower dose of tamsulosin.
